(19) United States
(12) Patent Application Publication
(10) Pub. No.: US 2011/0276123 A1
(43) Pub. Date: Nov. 10, 2011

(54) VASCULAR STENT DESIGN

(76) Inventors: Peter F. Davies, Haverford, PA (US); Juan M. Jimenez, Philadelphia, PA (US)

(21) Appl. No.: 12/744,871

(22) PCT Filed: Nov. 25, 2008
(86) PCT No.: PCT/US08/84761

§ 371 Date: Oct. 25, 2010

Related U.S. Application Data
(60) Provisional application No. 60/996,603, filed on Nov. 27, 2007.

(51) Int. Cl. A61F 2/82

(52) U.S. Cl. 623/1.15; 623/1.42

(57) ABSTRACT

This invention is directed to the design of radially expandable vascular stents to optimize hemodynamic flow characteristics that are favorable for the inhibition of stent-associated thrombosis, inflammation, and restenosis (neointimal formation) and that will reduce the risk of adverse events post-deployment.

### FIELD OF THE INVENTION

This invention relates to the design of radially expandable vascular stents to optimize hemodynamic flow characteristics that are favorable for the inhibition of stent-associated thrombosis, inflammation, and restenosis (neointimal formation) and that will reduce the risk of adverse events post-deployment.

### BACKGROUND OF THE INVENTION

In coronary arteries, at sites where atherosclerosis is present, there often occurs a stenosis that reduces blood flow to the myocardium and leads to angina or to an infarction. Deployment of one or more radially expandable vascular stents is a common procedure of choice in order to physically reopen stenotic regions of coronary arteries, i.e., to locally restore the diameter of the lumen, and enhance the flow of blood to the myocardium. However, restenosis, the re-formation of a neointima that re-narrows the arterial lumen, is a recurrent problem in ˜30% of patients receiving bare metal stents (BMS).

To counter restenosis, drug-eluting stents (DES) that release inhibitors of neointima formation over a period of weeks in order to inhibit restenosis were developed and subsequently approved by the U.S. Food and Drug Administration (FDA) in 2003 and 2004; they are in widespread clinical use. At present, there are five drug-eluting stents approved by the FDA in the United States: (1) TAXUS™ Express2™ Paclitaxel-Eluting Coronary Stent System, manufactured by Boston Scientific, Natick, Mass. (FDA approval Mar. 4, 2004), (2) CYPHER™ Sirolimus-eluting Coronary Stent, manufactured by Cordis Corporation, Miami Lakes, Fla. (FDA approval Apr. 24, 2003), (3) Endeavor™ Zotarolimus-Eluting Coronary Stent System manufactured by Medtronic, Minneapolis, Minn. (FDA approval Feb. 1, 2008), (4) Xience V Everolimus-eluting Coronary Stent System manufactured by Abbott Laboratories, Abbott Park, Ill. (FDA approval Jul. 2, 2008), and (5) TAXUS® Liberte® Paclitaxel-Eluting Coronary Stent System manufactured by Boston Scientific, Natick, Mass. (FDA approval Oct. 10, 2008) DES have proven effective in inhibiting neointimal formation, and hence restenosis, for extended periods.

Unfortunately, recent studies suggest a small but significantly increased risk of late stent thrombosis in DES patients that results, in the majority of cases, in death or myocardial infarction. In late 2006, the FDA expressed concern for the safety of DES, noting that a significant increase in the rate of death and myocardial infarction was observed in patients 18 months to 3 years after stent implantation. At a Dec. 7-8, 2006 meeting of the Circulatory System Devices Panel of the FDA, histological and morphological evidence was presented showing a greater incidence of inflammation and fibrin deposition on and between the stent struts in DES when compared to bare metal stents. These differences were also associated with significantly less re-endothelialization of DES and the retention of the stent strut at or near the surface of the artery (because of the inhibition of neointima formation that, in the case of bare metal stents, tends to grow over the strut). The FDA meeting that concluded on Dec. 8, 2006 resulted in a recommendation to issue new warnings to doctors and patients informing them that the safety of the devices has not been established.

An important fact relevant to this disclosure has emerged from investigation of the longer-term response of arteries to DES. In order to inhibit restenosis, DES inhibit the growth of neointimal tissue between and over the stent struts. Consequently, the stent struts may remain in indefinite contact with the flowing blood instead of being overgrown by the neointima as more readily occurs with BMS.

The use of bare metal stents (BMS) in coronary artery stenting is also widespread, and their use as an alternative to DES has increased following reports of late stent thrombosis with DES. In BMS, the peak risk for thrombosis occurs at and shortly after stent deployment and decreases over several days-to-weeks as new tissue fills in and the inter-strut neointima formation reduces the relative protrusion of the strut into the blood flow with eventual endothelialization of the neointima.

Atherosclerosis is an inflammatory disease of arteries that involves the participation of multiple vascular wall cells (endothelium, smooth muscle cells, resident immune cells) and infiltrating blood cells (monocyte-derived macrophages and other circulating blood cells). Advanced plaques often develop a pro-thrombotic surface in contact with the blood, resulting in thrombotic emboli or resident clots. Strong correlations have been observed between regions of separated flow (often termed “disturbed flow regions”) in the cardiovascular system and arterial wall sites prone to the development of atherosclerotic lesions. Here, the local vessel geometry (e.g., near branches, bifurcations and curvatures of arteries) causes the flow to locally separate from the bulk fluid trajectory.

The inventors propose that current stent design and cross-sectional geometry largely ignores the flow implications of stent geometry of the strut-blood and strut-vessel interfaces upon thrombosis and inflammation. Learning from studies of atherogenesis in relation to blood flow disturbances induced by naturally-occurring complex vessel geometries, the inventors hereof have discovered that the current stent strut geometry creates local regions of flow separation (flow disturbances) that lead to a pro-thrombotic and pro-inflammatory environment at and around the stent struts. Similar regions occur naturally in the arterial circulation at branches, bifurcations and sharp curvatures where separated flow within the region occurs as a result of the geometric changes in the vessel, and such regions are susceptible to atherosclerosis and its associated thrombotic and inflammatory risks. A similar environment of atherosclerotic risk exists around the deployed stent where the nonstreamlined strut cross-section, characteristic of stents currently in clinical use, creates flow separation regions at the leading and trailing edges of the strut, the trailing edge effect being particularly prominent. Of particular note is that exposure of blood to the stent may continue for months after deployment of DES, extending the thrombotic and inflammatory risks.

The cross-sectional profile of currently approved stents is nonstreamlined (rectangular, circular, and trapezoidal) with some slight rounding of the edges for non-circular stent struts. Blood flowing over such profiles undergoes a significant region of flow separation, particularly downstream of each strut, that is a favorable local environment for blood coagulation even in the presence of endothelium in which flow separation induces pro-thrombotic and pro-inflammatory endothelial cell phenotypes. Thus, the present stent configurations do not accommodate a design that minimizes flow disturbances as the blood passes over the stent struts. By largely ignoring the hemodynamic interactions between the flowing blood and the stent surface profiles, a higher risk of stent-induced thrombosis persists while the stent is at or near the artery surface. For DES, this period may extend indefinitely; for BMS that are overgrown by neointima, re-endothelialization appears to occur more quickly thereby reducing the risk of thrombosis. Therefore, thrombosis risk for BMS is greatest during the first weeks to months after deployment. It is noteworthy that both BMS and DES have a similar incidence of stent thrombosis during the first 9 months despite aggressive anti-coagulant therapy. For DES, current recommendations include the indefinite continuation of anti-coagulant therapy as long as tolerated by the patient. The designs proposed in the invention address stent thrombosis associated with both DES and BMS.

The inventors have reported differential transcript profiles of endothelial cells in regions of flow disturbance vs. regions of undisturbed flow in large arteries and heart valves in a swine animal model. See, P. F. Davies et al., A spatial approach to gene expression profiling: mechanotransduction and the focal origin of atherosclerosis, Trends in Biotechnology, 17:347-351 (1999); A. G. Passerini et al., Coexisting pro-inflammatory and anti-oxidative endothelial transcription profiles in a disturbed flow region of the adult porcine aorta, Proc. Natl. Acad. Sci. USA, 101:2482-2487 (2004); and C. A. Simmons et al., Spatial heterogeneity of endothelial phenotypes correlates with side-specific vulnerability to calcification in normal porcine aortic valves, Circulation Research, 96:792-799 (2005), the disclosure of each of which is incorporated herein in its entirety. Furthermore, the inventors have described differential post-translational modifications of important endothelial proteins in comparative disturbed and undisturbed flow regions in vivo. See R. Magid et al., Endothelial protein kinase C isoform identity and differential activity of PKCζ in an athero-susceptible region of porcine aorta, Circulation Research, 97:443-449 (2005).

Separated flow regions often develop transient vortices and are characterized by complex spatial and temporal flow non-uniformities, flow reversal, lower fluid flow velocities than those observed in the mainstream, and lower hemodynamic shear stresses than those present in attached flow regions. There is a large literature from the inventors [See, e.g., P. F. Davies, Flow-mediated endothelial mechanotransduction, Physiological Reviews, 75:519-560 (1995)] and elsewhere demonstrating that surface forces such as shear stresses are sensed by the local endothelial cells, and it is generally accepted that the endothelium is important for the susceptibility or protection of atherosclerosis-prone regions of arteries via its interactions with the local flow environment.

U.S. Pat. No. 5,718,713 to Frantzen purports to teach a surgical stent formed of stent segments having a streamlined contour. The inner surface of each segment (the surface over which the blood flows), namely the inner leading region and the inner trailing region, has a greater curvature relative to the curvature of the outer surface of each stent segment (the surface in contact with the vessel wall). Thus, the inner surface purportedly does not present any abrupt transition in flow for bodily fluids passing thereover, particularly when the stent segment is aligned circumferentially with bodily fluid flow passing adjacent the inner surface from a leading inner edge to a trailing inner edge. However, while the inner surface of the struts may indeed have a smoothed contour, it is clear that the curvature disclosed exceeds that required to mitigate or eliminate flow separation at physiological Reynolds numbers and that the geometry of the strut surface relative to the lumen wall will still result in significant flow separation of the blood as it passes over the strut.

U.S. Pat. No. 6,685,737 to Pacetti purports to teach a stent design that minimizes the disturbance of blood flow and the trauma caused by the stent to the vessel in which it is implanted. However, while the geometry of the disclosed stent struts have an outer surface that may indeed reduce the injury and inflammation to the vessel wall, there is no indication that the geometry of the inner surface of the struts reduces flow separation of the blood as it passes over the struts as intended.

There is clearly compelling evidence for a cause-effect relationship between flow disturbance and a propensity for pro-inflammatory, pro-thrombotic vascular responses. It is desirable, therefore, to provide an improved design for DES that avoids or significantly reduces the incidence of inflammatory, thrombotic vascular responses in addition to restenosis. For BMS, optimal strut design is desirable for similar reasons, as streamlining is proposed to reduce the thrombosis risk that occurs early after deployment and may also reduce the severity of neointimal hyperplasia associated with BMS.

In carotid arteries that supply blood to the brain, severe atherosclerosis may narrow the vessels reducing blood flow or causing blood clots to form at the plaque sites. Often, thrombotic emboli detach resulting in a stroke or a series of transient episodes of ischemia in the brain. For patients with high risk for endarterectomy, the deployment of a stent after angioplasty is an alternative clinical option. For such circumstances, optimal stent strut design is desirable for the same reasons considered above as they relate to coronary stenting.

Similarly, where peripheral vascular disease renders other arterial sites suitable for stenting, the design of optimal stent strut geometry to minimize flow separation is desirable for the same reasons considered above as they relate to coronary and carotid stenting.

### SUMMARY OF THE INVENTION

The inventors hereof have discovered that the post-deployment geometry of the stent is an important predictor of the predisposition to thrombotic and other pathological changes. As shown in FIGS. 11A-D, flow disturbance is understood to contribute to arterial pro-thrombotic and pro-inflammatory tendencies when the sectional profile encountering the flow is nonstreamlined. In the field of fluid mechanics, it is well known that bluff (blunt) or nonstreamlined bodies are more prone to experience fluid flow separation, even at moderate Reynolds numbers. For laminar flows, this happens earlier than for turbulent flows, resulting in larger separated flow regions. In general, the flow regime in the cardiovascular system can be described as unsteady laminar flow, and flow separation associated with the strut is accompanied by low shear stress distributions at the arterial wall. Thus, the design of struts disclosed by the inventors incorporates fluid and solid mechanics principles while taking into account the local pathophysiology.

Accordingly, it is one object of the present invention to minimize or eliminate local flow disturbances that lead to a pro-thrombotic and pro-inflammatory environment at and around the struts of a radially expandable surgical stent.

It is another object of the present invention to provide a stent with a streamlined inner surface contour and cross-sectional geometry where the strut-blood interface and strut-vessel interface create a fluid dynamic environment that is more conducive to inhibition of thrombosis and inflammation.

In accordance with these and other objects of the invention, one embodiment of the invention provides a stent whose struts have an inner surface contour design and cross-sectional geometry that streamline the strut-blood and strut-vessel interfaces to create a fluid dynamic and pressure distribution environment that is more conducive to inhibition of thrombosis and inflammation.

In one embodiment of the invention, a stent, for example a BMS or a DES or a degradable stent, provides attached or minimally separated blood flow therethrough, the stent comprising one or more struts, each having an inner surface contour that provides attached or minimally separated blood flow thereover. The contour of the strut inner surface, i.e., the surface over which the blood flows, has, in the bulk flow direction, a leading end and a trailing end and a continuous surface in between having a varying slope throughout. For simplification, this may be described as a strut having a cross-sectional geometry longitudinally disposed thereon, wherein the leading subsection affects a directional change while keeping the blood flow attached through a favorable pressure gradient over the leading subsection of the strut, the trailing subsection affects a directional change while keeping the blood flow attached, and a midsection disposed therebetween, thereby providing a favorable geometry to ensure that the flow follows the stent geometry without separation.

The surface of the leading region is defined by a curve with infinite points. A tangential line at each point has a finite slope. The slopes for the tangential lines start at a positive slope and transition smoothly to a zero slope as the leading region approaches the middle region. The slope at each point for the middle region, which may exist as a single point or many, equals zero. The surface of the trailing region is defined by a curve with infinite points. A tangential line at each point has a finite slope. The slopes for the tangential lines start with a value of zero and smoothly transition towards a negative finite slope as the points approach the trailing edge.

The invention also provides a method of ensuring attached flow without flow separation through a BMS or DES or a degradable stent, comprising implanting in a predetermined arterial location a stent, for example a BMS or a DES or a degradable stent, comprising at least one strut having an inner surface contour with, in the direction of blood flow, a leading edge and a trailing edge and a continuous inner surface in between having varying slope throughout. This may be described as a strut having a cross-sectional geometry longitudinally disposed thereon, wherein the leading subsection affects a directional change while keeping the blood flow attached through a favorable pressure gradient over the leading subsection of the strut, the trailing subsection affects a directional change while keeping the blood flow attached or minimally separated, and a midsection is disposed therebetween, thereby providing attached or minimally separated blood flow over the cross-sectional length of the stent.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The above and other objects and advantages of the invention will be apparent upon consideration of the following detailed description, taken in conjunction with the accompanying drawings, in which the reference characters refer to like parts throughout and in which:

FIGS. 1A and 1B shows perspective views of portions of two examples of radially expandable surgical stents in an open, expanded position, FIG. 1A showing a helical coil stent structure, and FIG. 1B showing an expanded lattice stent structure;

FIG. 2 shows a first embodiment of a cross-sectional view of an individual stent coil element or strut;

FIG. 3 shows a front view of an individual stent coil element or strut such as at or near the apex of an expanded lattice stent, for example as shown in FIG. 1B;

FIGS. 4A-C show flow simulations illustrating the difference in effect of width, w, to height, h, ratios (aspect ratios) of rectangular strut profiles of 2:1, 4:1, and 8:1, for a 10w inter-strut spacing, which is in the interstrut distance range for typical commercial stents;

FIG. 5A-C shows flow about a three different circular arcs with a width to height ratio of 2:1, 4:1, and 8:1, for a 10w inter-strut spacing, which is in the interstrut distance range for typical commercial stents with the 4:1 and 8:1 aspect ratio circular arc struts illustrating the elimination of flow separation in this embodiment of streamlining;

FIGS. 6A-D show examples of stent strut cross-sectional configurations having a peak width to height ratio of 8:1;

FIGS. 7A-D show examples of stent strut cross-sectional configurations of FIGS. 6A-D but having a peak width to height ratio of 4:1; and

FIGS. 8A-D show examples of stent strut cross-sectional configurations of FIGS. 6A-D and FIGS. 7A-D but having a peak width to height ratio of about 2:1.

FIGS. 9A-D show different embodiments of stent strut cross-sectional configurations, each having a width to peak half-height ratio of 8:1, which are analogous to those in FIGS. 6A-D but symmetric about a line from the leading edge to the trailing edge.

FIGS. 10A-D show the same examples of stent strut cross-sectional configurations as in FIGS. 9A-D but where the width to peak half-height ratio was decreased to 4:1.

FIGS. 11A-D show: A. In the normal artery wall the anticoagulant properties of the endothelium help maintain hemostatic balance by the contribution of anti-coagulant properties (secreted and surface-presented) to the blood/cell interface. Blood contains multiple pro-coagulant proteins as well as natural anti-coagulants that together with the endothelium normally maintain a non-coagulation state. ADP, adenosine diphosphate; TF, Tissue Factor; vWF, von Willebrand Factor; PGI₂, prostacyclin; TFPI, Tissue Factor Pathway Inhibitor; TPA, Tissue Plasminogen Activator; TM, Thrombomodulin. B. The deployment of currently available commercial stents creates flow separation in the proximal and distal regions of the stent relative to the blood flow. Procoagulant conditions are greatly increased around the stent strut by the following: (i) accelerated flow over the strut edges generates shear stress peaks at magnitudes that can activate platelets some of which will enter the distal flow separation zone, (ii) low flow velocities in the separation region retain activated platelets and procoagulant plasma factors that reach critical concentrations for assembly of the coagulation cascade, (iii) the removal of endothelium during angioplasty and stenting eliminates key anticoagulant protective mechanisms and exposes a thrombogenic surface (extracellular matrix, residual lesion material) for platelet adhesion, aggregation and, when the clotting cascade activates, thrombus formation. The flow separation resulting from this design of stent strut represents a ‘micro-reaction chamber’ weighted towards pro-thrombotic pathways. Furthermore, (iv) low flow velocity and low shear stress inhibit re-endothelialization of the vessel. C. A modest streamlining of the strut cross-sectional profile eliminates flow separation and maintains uninterrupted high flow velocity that greatly reduces the probability of pro-thrombotic reactants reaching critical levels despite the absence of endothelium. Platelets adhere to the de-endothelialized surface but the higher flow velocities inhibit aggregation (e.g. ADP release from platelets is rapidly diluted, reducing its effectiveness for chemical activation of additional platelets). Undisturbed flow also favors re-endothelialization of the stented region. D. Restitution of an endothelialized surface restores the anti-coagulant checks and balances of the endothelium to provide further protection against stent-related thrombosis.

FIG. 12 shows Cross-sectional stent strut geometries with different aspect ratios, AR=width to height (w:h).

FIG. 13 shows Coarsest grid spacing mesh in the vicinity of a 2:1 aspect ratio rectangular stent strut.

FIG. 14 shows Shear stress per unit length (τ*_{w}), —∘—, variation for a 2:1 AR rectangular strut as a function of grid spacing. Theoretical shear stress per unit length, ⋄, approximation calculated using Richardson extrapolation for the hypothetical case of zero grid spacing.

FIG. 15 shows Wall shear stress and shear rate distributions corresponding to (a) rectangular and (b) circular arc stent struts for aspect ratios, AR=2:1, - - - , 4:1, - - - and 8:1, . . . .

### DETAILED DESCRIPTION OF THE INVENTION

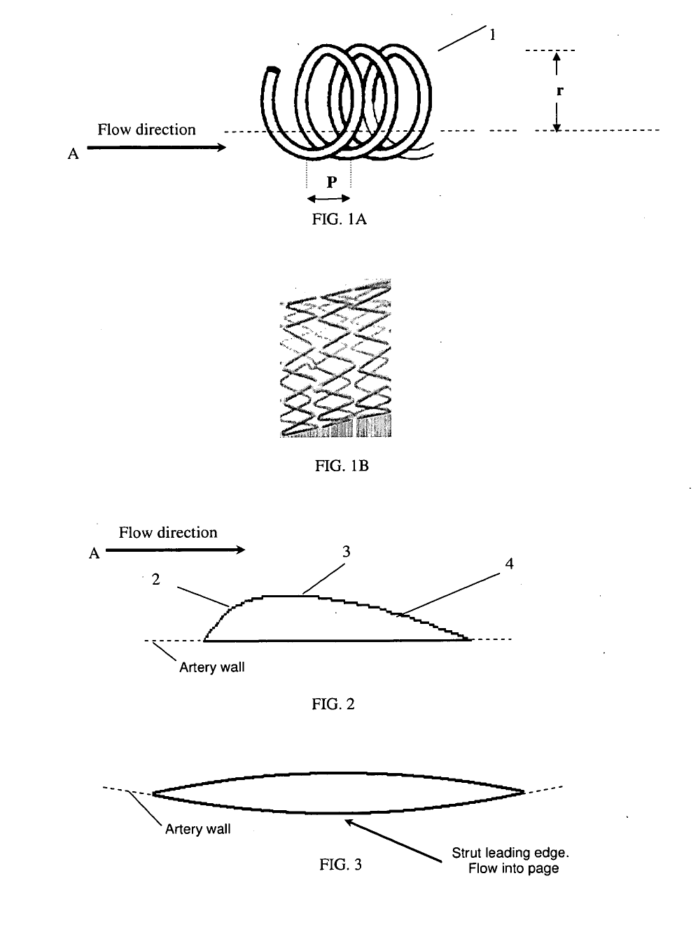

FIG. 1 shows the leading portion of one embodiment of a radially expandable surgical stent 1 in an open, expanded position, as it would be when implanted within a body lumen (body lumen not shown). The stent, when deployed, has a generally tubular or rounded-rectangular configuration and is typically formed from multiple stent elements or struts, which, in FIG. 1A, are formed in a helical arrangement and in FIG. 1B are formed in a lattice pattern. It should be noted that this invention is not limited to any particular design or pattern of stent construction, and may be used whether the stent has a helical shape, has a lattice pattern or has any other configuration of struts, whether expandable or not. In virtually every embodiment of a stent, the stent has individual structural elements intended to lie along the circumference of the lumen, either in a circumferential direction (90° to the direction of blood flow), a longitudinal (axial) direction (parallel to the direction of blood flow) or some other direction in between.

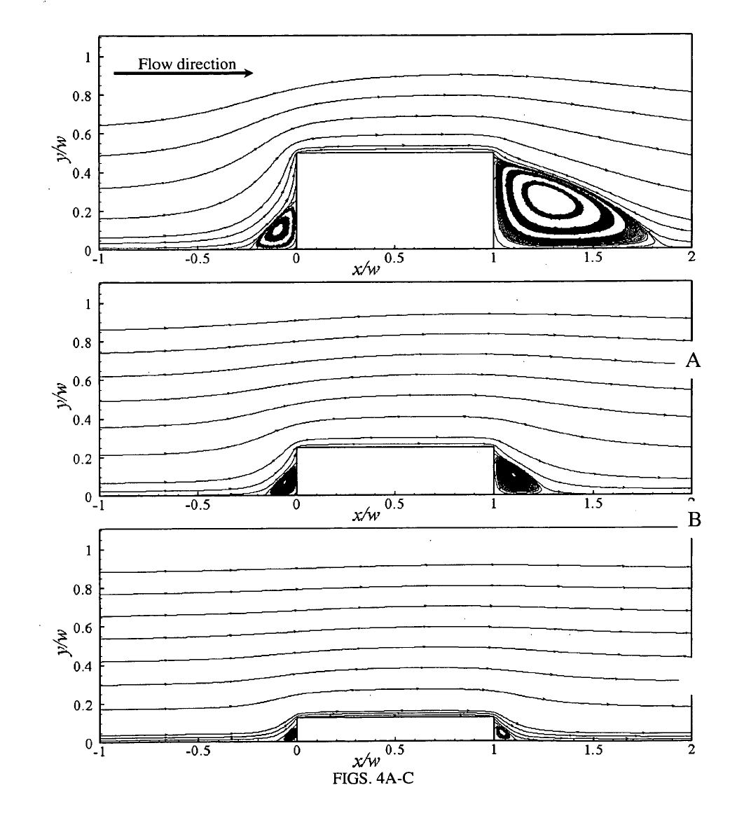

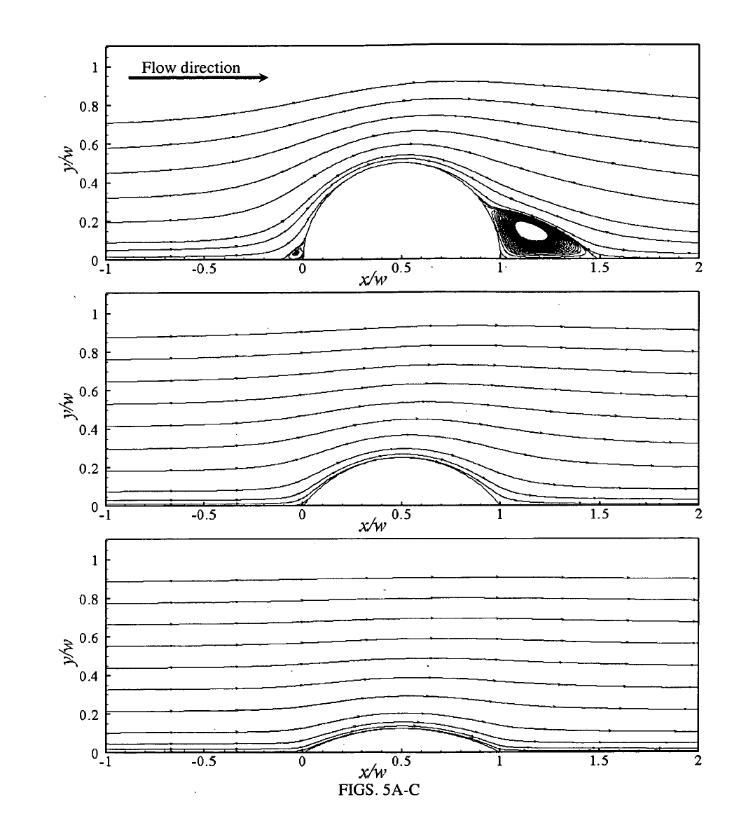

In a first embodiment, discussions herein referring to the surface contour and cross-sectional shape of stent 1 refer to the surface contour and cross-sectional shape of the individual stent element or strut, one embodiment of which is shown in FIG. 2, which is a coil element in the embodiment of FIG. 1A and a lattice configuration in the embodiment of FIG. 1B. It should be noted that the term “cross-section” as used herein with regard to the cross-section of the strut of a stent, such as shown in FIG. 2, refer to sections taken of the strut in the longitudinal direction of the stent when deployed, namely in the direction of blood flow through the lumen or vessel (left to right, see arrow A in FIG. 1). Due to the helical nature of the stent shown in FIG. 1A, and due to the fact that in other stent configurations the struts of a stent most likely not oriented precisely normal to the blood flow (e.g., FIG. 1B), this section will necessarily not be the transverse cross-section of the stent strut, i.e., at 90° to the strut direction. Depending upon the direction of the strut with respect to the blood flow, the angle of this cross-section with respect to the direction of the deployed strut will vary. For the embodiment shown in FIG. 1, this could depend upon the number of coils or the strut lattice arrangement and the tightness of the coils or struts within the stent, namely the number of coils or struts turns per unit distance.

In the case of stent lattice designs, depicted in FIG. 1B and well known in the art, any connecting struts, arms or segments will also be of streamlined surface contour and cross-section, irrespective of the predicted angle of the blood flow direction. For instance, in the extreme example of a stent strut or link that extends longitudinally with respect to the lumen or vessel and to the blood flow within it, the streamlining will apply to the longitudinal disposition of the strut. Furthermore, as shown in FIG. 3, the contour of the outer surface of this strut across its length in the circumferential direction, i.e., the surface adjacent to the vessel wall, will be approximately matched to the curvature of that lumen. FIG. 3 illustrates a view of such a design for the apex of an expanded lattice or the forward edge of a longitudinal connecting strut commonly employed in expandable lattice stents.

FIG. 2 shows a first embodiment of the cross-section of the strut of stents 1A and 1B. As shown in FIG. 2, the upper surface contour of the strut cross-section, namely the inner stent surface, i.e., the surface over which the blood flows, exploits solid and fluid mechanics design principles to minimize the disturbance of blood flow in the vessel in which the stent is implanted, and the bottom surface contour, namely the outer stent surface, i.e., the surface adjacent to the inner surface of the body lumen within which it is deployed, has smooth leading and trailing edges with a relatively flat surface. In order to apply a uniform normal force to the underlying vessel wall, the outer (bottom) surface of the strut follows the contour of the blood vessel transverse to the flow direction as seen in FIG. 3 to avoid pressure points that may be detrimental to the local healing.

The top and bottom surfaces come together at smooth edges. By distributing the load and incorporating hydrodynamic principles to the inner surface and edges of the stent, (i) flow disturbances, which create greater risk for coagulation and inflammatory responses, are reduced, thus reducing the risk of both early and late stent thrombosis and inflammation, (ii) the shear rates and shear stresses that attain sharp peaks at the proximal and distal edges of rectangular and minimally streamlined struts are reduced below levels known to activate platelets in the blood (see Examples), and (iii) more favorable conditions for endothelialization of the strut surface and adjacent vessel wall are introduced.

In a first embodiment, as shown in FIG. 2, wherein the direction of blood flow through the vessel is shown by arrow A, the strut will preferably have an upper surface (i.e., inner surface of the stent geometry) whose surface contour has a cross-section geometry longitudinally disposed thereon, wherein the subsection that is leading with respect to blood flow affects a directional change while keeping the blood flow attached through a favorable pressure gradient over the leading subsection of the strut cross-section, the subsection that is trailing with respect to blood flow minimizes the probability of flow separation, and a midsection contour is disposed therebetween, the whole contour providing attached or minimally separated flow over the inner surface of the stent strut.

In the first embodiment, the leading (upstream) section 2, i.e., the portion of the strut that is first contacted by the blood flowing over the strut, will follow a hydrodynamically streamlined contour to allow the fluid flow direction to change gradually while introducing a favorable pressure gradient over the front face of the strut. In a preferred embodiment, the surface contour has a continuously varying slope throughout to provide a smooth leading surface. This avoids the flow separation experienced by nonstreamlined strut cross-sections.

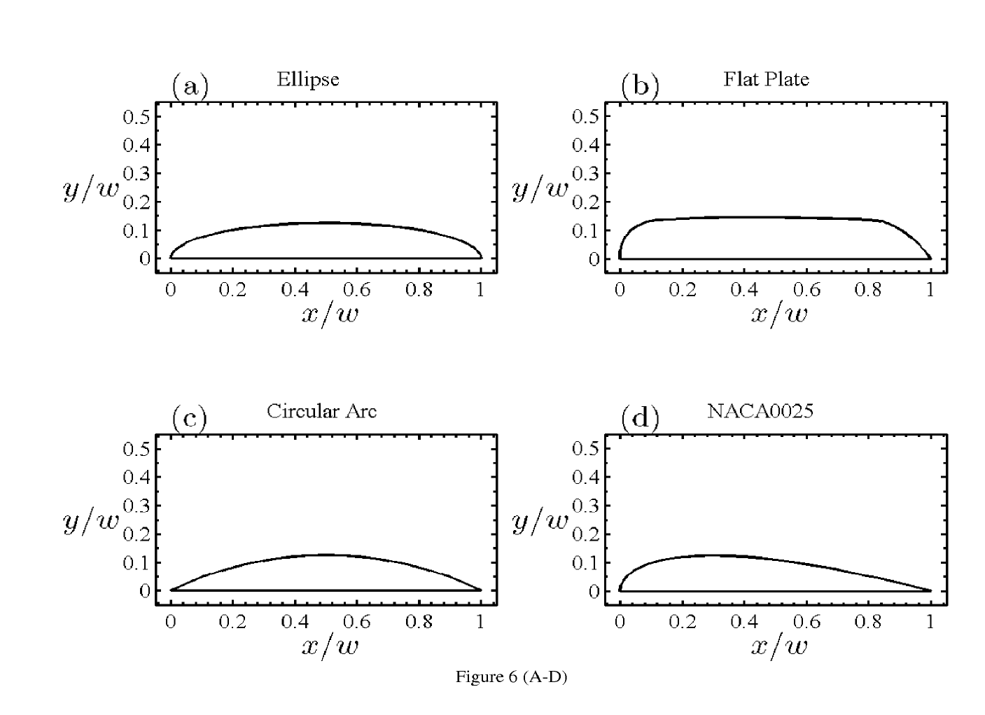

The trailing (i.e., downstream) section 4 of the strut cross-section will be streamlined, allowing for the gradual change of the flow direction and avoiding sudden changes in direction, which are responsible for flow separation in low momentum flows. The streamlined geometry will help minimize the probability of flow separation and the adverse physiological consequences to the vascular tissue. In one preferred embodiment, the trailing section 4 transitions smoothly with respect to the lumen surface as does the leading section 2, and in another preferred embodiment, the trailing section 4 is more streamlined, i.e., transitions more smoothly with respect to the lumen surface, than is the leading section 2. Examples of these embodiments are illustrated in FIGS. 6A-D, 7A-D and 8A-D (discussed below).

The mid-section 3 of the strut cross-section, i.e., the middle portion between the leading and trailing edges, is flat and is contiguous with the leading and trailing regions. The mid-section may exist as a single point or may be extensive. When the stent is fully deployed, an outward force in the radial direction keeps the stent in place through friction with the lumen wall. This force is applied through the outer surface of the stent that is in contact with the inner surface of the blood vessel. The pressure applied is constant, since it is dictated by the material properties of the deployed stent. The force experienced by the tissue in contact with the stent is dependent on the area over which the normal force is distributed. If the contact surface is increased through the elongation of the strut cross-section, then the normal force to which the tissue will be subjected will decrease (since pressure=force/area). Thus, the contact surface area of the strut will be optimized to allow for the appropriate level of tissue exposure to the blood, while minimizing the normal force experienced by the tissue when the stent is fully deployed.

In one embodiment, blood is best considered as a suspension of red and white blood cells and platelets in liquid plasma. Accordingly and in one embodiment, lower velocities and/or the flow three-dimensionality, characteristics of disturbed flow, will bring platelets, cells, etc. directly to the vessel wall creating sedimentation of suspended blood cells that accelerate thrombus formation and growth. The strut profile described herein eliminates or minimizes locally disturbed flow and substantially reduces the residence time of blood cells and particles.

In another embodiment, disturbed flow encompasses steep spatial and temporal gradients of shear forces, and multi-directional hemodynamic forces. These conditions, unfavorable to the biology of the vessel wall and pro-coagulative/pro-inflammatory in nature, are minimized or eliminated by the proposed stent strut design. In another embodiment, platelets entering a disturbed flow region in an activated state contribute to pro-thrombotic conditions by interactions with other pro-coagulative elements. By substantially eliminating disturbed flow through the use of the streamlined strut profile described herein, the devices described herein substantially reduce the probability of a patient developing restenosis when treated with the bare metal stents or DES stents described herein.

By using a multi-segmented streamlined geometry for the stent strut, the inventors intend to exploit the streamlined geometry of a hemodynamic hydrofoil (analogous to an airfoil in aerodynamics) with the structural integrity provided by an elongated strut cross-section. Through computational fluid dynamics modeling and/or physical experiments, the effect of the stent strut designs upon the blood flow can be optimized. While such well-known fluid dynamics principles have been used to perform similar model analyses to demonstrate the predictive effect upon flow of the number and positioning of the struts of a stent (of existing design), consideration of the strut profile detail has not heretofore been investigated in relation to mitigation or elimination of flow disturbances and the implications for optimal inter-strut positioning.

A distinctive aspect of the invention is the geometrical dimensions of the strut cross-section that are utilized to achieve the most desirable flow characteristics, namely achieving a favorable pressure gradient at the leading section of the strut and minimizing flow separation at the leading and trailing regions thereof. It is shown that flow separation is minimized by incorporating a streamlined design of the leading and trailing regions of the cross-sectional shape of the stent strut, namely the degree of curvature thereof. Profiles having a more gradual curvature at the leading and trailing regions of the stent are more preferable from a hydrodynamic standpoint.

The inventors have also discovered that the ratio of the width of the strut cross-section to the height of the base of the strut cross-section over which the blood flows also contributes significantly to the hydrodynamic flow characteristics of the stent profile. Thus, in one embodiment, a wider and lower stent strut profile will perform better hydrodynamically than will a thinner and/or higher stent strut profile.

In another embodiment, this “width to height” ratio (aspect ratio) is preferably greater than about 4:1; with current manufacturing and biological limitations this ratio can likely be increased. The inventors recognize that new materials in stent manufacture may allow this range of ratios to be further increased.

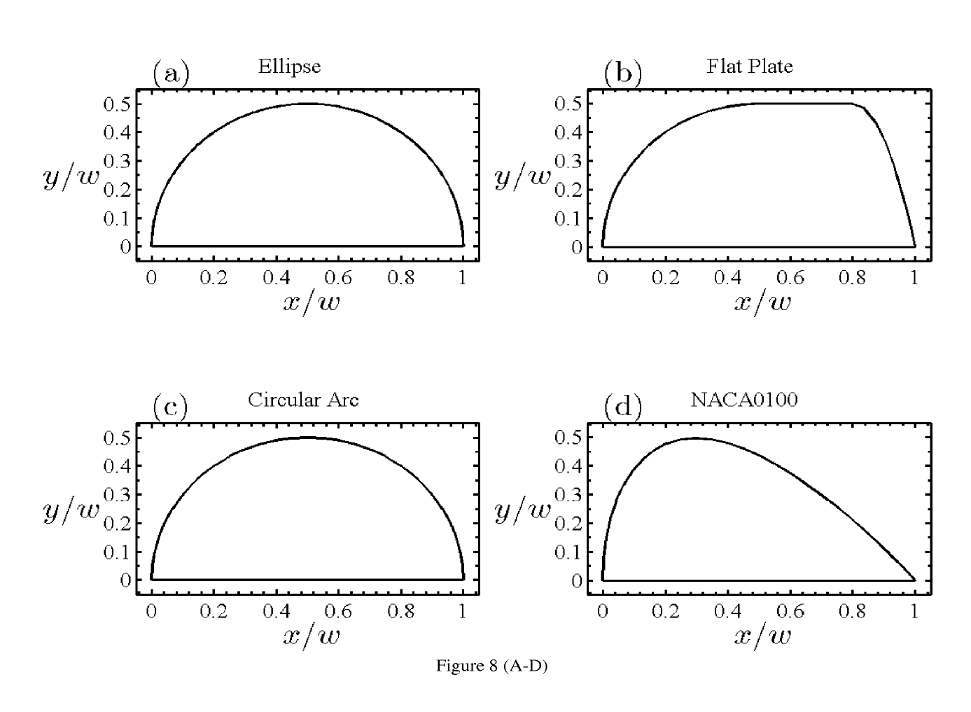

FIGS. 4A-C show numerical flow simulations illustrating the difference in effect of strut width to height ratios of 2:1 (FIG. 4A), 4:1 (FIG. 4B) and 8:1 (FIG. 4C) upon flow separation (flow left to right). In these embodiments, the cross-sectional shapes are rectangular and not streamlined, i.e., they have flat leading and trailing faces that are oriented perpendicular to the direction of blood flow in the lumen, which is a widely-used cross-sectional configuration. In this simulation, the only independent variable is the height of the strut. In one embodiment, the strut is extended to double peak height symmetric to a line extended from the leading edge to the trailing edge. Variations of the lower surface curvature and height that deviate from symmetry are also encompassed herein, as long as the upper surface is in contact with the blood meets the streamlined definition, i.e. absence of flow separation.

In flow simulations FIGS. 4A-C, the Reynolds numbers for these simulations based upon the inner diameter of the vessel was 400, which is in the upper range of coronary arterial flow. Also, as stated, all variables were kept constant except variation of the strut height from 100 μm (FIG. 4A) to 50 μm (FIG. 4B) to 25 μm (FIG. 4C), with a strut width of 200 μm in each case. The figures show streamlines denoting the path of fluid flow. Testing of these cross-sectional configurations was performed by simulating flow over the stent structure and measuring flow disturbances at the leading and trailing edges. It is evident from these plots; that as the height of the strut is decreased, the size of the separation region downstream of the leading and trailing edges decrease in a nonlinear fashion. In each of these simulations, inter-strut spacing was 2 mm, which was much larger than the size of the separation region following the trailing edge, such that inter-strut spacing played no primary role in flow separation of these simulations.

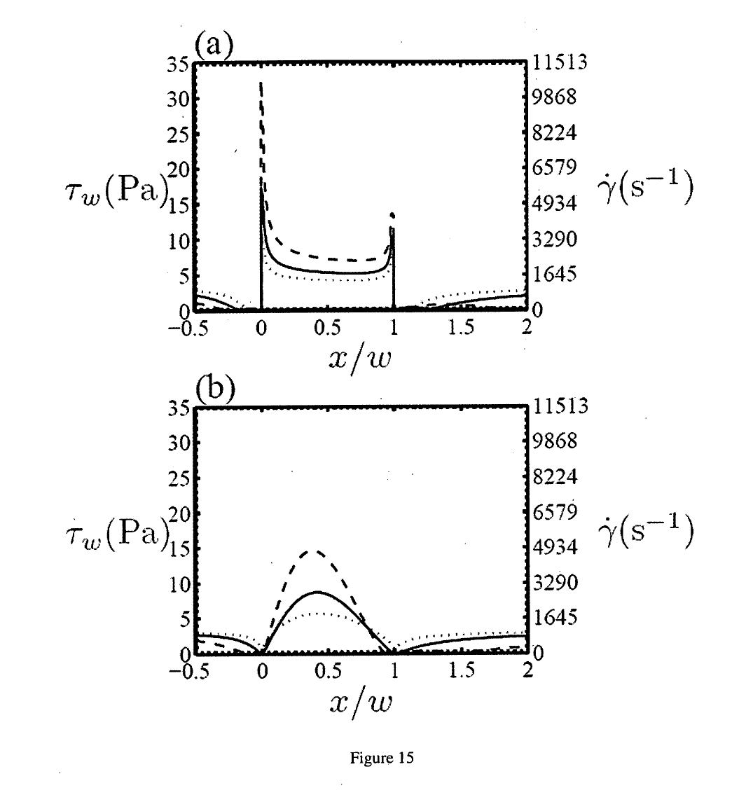

Nonstreamlined stent struts such as rectangular cross-section geometries can be modified by decreasing the height, h, and consequently lessening the effect of h on the flow field. However, the recirculation zone persists maintaining the potential to form a nidus for thrombi (FIG. 4a-4c). The decrease in thickness not only decreases the size of the recirculation volume, but also decreases the area of the endothelium exposed to disturbed flow thus increasing the probability of endothelialization of adjacent tissue. The peak shear stresses and the shear stress values over the strut surface decline with decreasing thickness of the rectangular strut, (FIG. 15a). The lower shear stress values observed for the 4:1 and 8:1 AR rectangular cross-section struts are more conducive towards endothelialization of the strut surface despite the retention of a recirculation zone (FIG. 4a-4c). These considerations contribute in one embodiment, to the improved outcome for thinner stent struts observed clinically.

In one embodiment, the term “Reynolds number” refers to the function Rₑ=DvP/μ used in fluid flow calculations to estimate whether flow through a lumen is streamline or turbulent in nature. D is the inside lumen diameter, v is the average velocity of flow, P is density, and μ is the viscosity of the fluid. Reynolds number values much below 2100 correspond to laminar flow, while values above 3000 correspond to turbulent flow. In another embodiment, while laminar in nature, blood which is a suspension demixes and activates platelets as a stress response at Rₑ greater than about 400.

FIGS. 5A-C show flow about a circular arcs with aspect ratios of 2:1 (FIG. 5A), 4:1 (FIG. 5B), and 8:1 (FIG. 5C). It can be observed in FIGS. 5B-C that the fluid flow is traveling smoothly from left to right over these first embodiments, a circular arc strut, without flow separation. The streamlines denote the path of fluid particles. It is evident from FIGS. 5B-C that the flow has remained attached over the entire strut surface in contrast with that observed in FIGS. 4 and 5A, where flow separation occurred upstream and downstream of the rectangular and 2:1 circular arc cross-sectional struts, respectively.

From FIGS. 5B-C, it can be concluded that flow over a streamlined geometry remains attached in this embodiment and does not separate at physiological Reynolds numbers.

It should be understood that optimization of a streamlined geometry will depend on multiple factors such as but not limited to; construction material, lumen diameter, location of the implant, lumen wall thickness and the like.

A typical current stent strut is about 100 microns (μm)+/−20 high×wide in a square or near square cross-section. However, it is believed that the optimum designs will tend toward a lower height and wider section, e.g., 50 μm×200 μm, with of course streamlining, as discussed below.

FIGS. 6A-D show different embodiments of possible stent strut cross-sectional configurations, each having a peak width to height ratio of 8:1. In each of these examples, the width of the base of the cross-sectional shape is 1.0 unit long and the peak height is 0.125 units high. Each cross-section configuration will exhibit different effects of minimizing flow separation (flow direction from left to right).

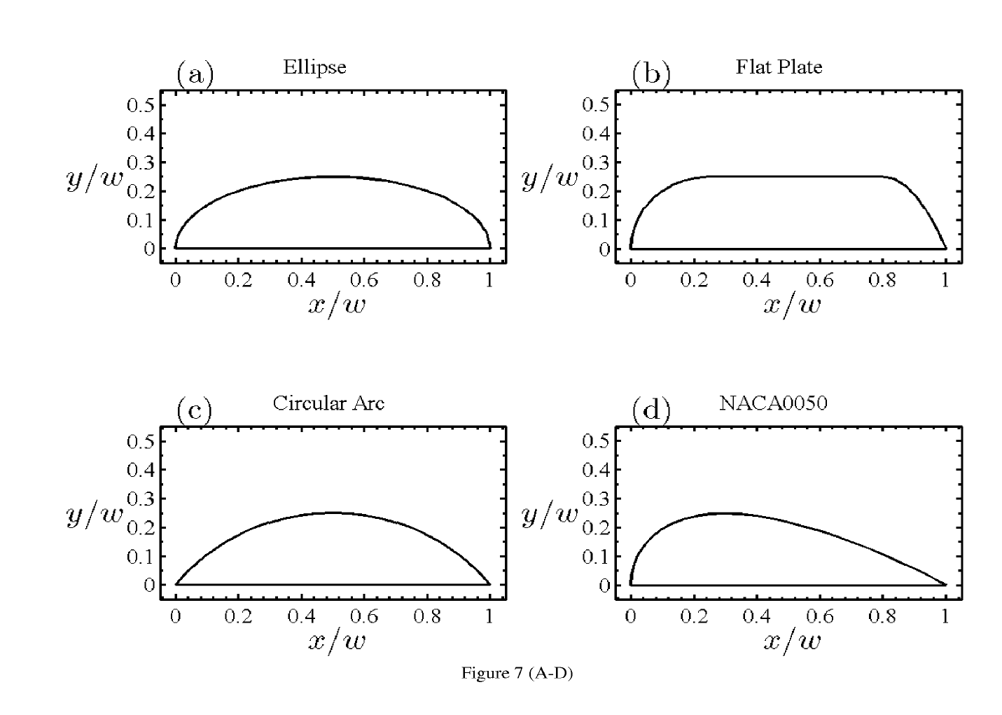

FIGS. 7A-D show the same examples of stent strut cross-sectional configurations as in FIGS. 6A-D but where the peak width to height ratio was decreased to 4:1, i.e., the height was proportionally increased to 0.25 units high, while the width of the base of the cross-sectional shape was kept at 1.0 unit long. It is not expected that the relative flow performance of the stent strut cross-sectional configurations will remain the same from FIGS. 6A-D to FIGS. 7A-D for the same Reynolds number since the advantages of streamlining have been decreased when the height was doubled.

FIGS. 8A-D show the same examples of stent strut cross-sectional configurations as in FIGS. 6A-D but where the peak width to height ratio was further decreased to 2:1, i.e., the height was proportionally increased to 0.5 units high, while the width of the base of the cross-sectional shape was kept at 1.0 unit long. Naturally, as can be seen by the differences in the slopes of the leading and trailing regions, simply increasing the width to height ratio will have a significant effect upon the slope of each of the leading and trailing regions. However, it is again not expected that the relative flow performance of the stent strut cross-sectional configurations will remain the same from FIGS. 6A-D to FIGS. 8A-D for the same Reynolds number since the advantages of streamlining have been decreased when the height was quadrupled.

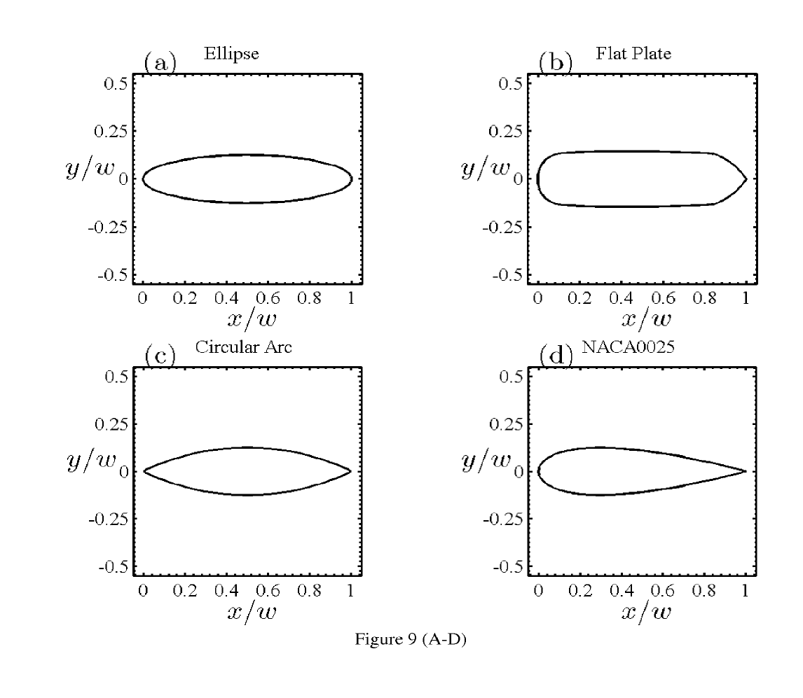

FIGS. 9A-D show different embodiments of possible stent strut cross-sectional configurations, each having a width to peak half-height ratio of 8:1, which are analogous to those in FIGS. 6A-D but symmetric in FIGS. 9A-D. This variant design is intended to take advantage of displacement of a soft vessel wall matrix by the lower strut surface thereby restoring an upper blood/stent interface geometry similar to the asymmetric designs shown in FIG. 6 after deployment. In each of these examples, the width of the base of the cross-sectional shape is 1.0 unit long and the peak height is 0.250 units high and the peak half-height is 0.125 units. Each cross-section configuration will exhibit different effects of minimizing flow separation (flow direction from left to right). We intend that this design will also allow for unequal curvatures of the upper and lower surfaces of the strut, i.e. an absence of symmetry.

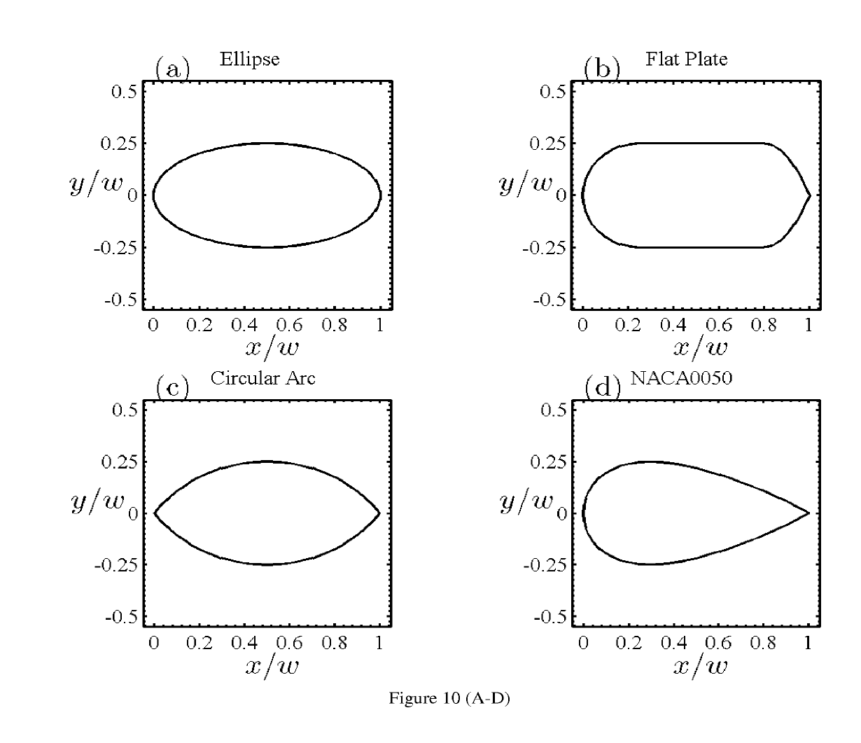

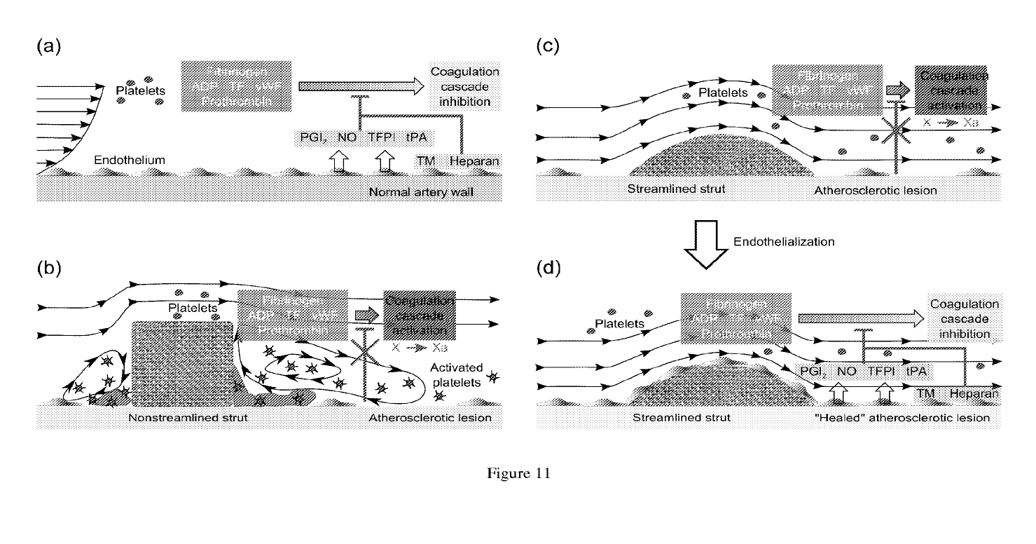

FIGS. 10A-D show the same examples of stent strut cross-sectional configurations as in FIGS. 9A-D but where the width to peak half-height ratio was decreased to 4:1. It is not expected that the relative flow performance of the stent strut cross-sectional configurations will remain the same from FIGS. 9A-D to FIGS. 10A-D for the same Reynolds number since the advantages of streamlining have been decreased when the height was doubled. We intend that this design will also allow for unequal curvatures of the upper and lower surfaces of the strut, i.e. an absence of symmetry.

Limitations in the material properties of stents will likely contribute to the optimal shape of the stent strut and its flow characteristics. However, it is believed that sufficient streamlining can be achieved using materials currently employed in stent manufacture.

The skilled practitioner will readily appreciate that maintenance of an optimized flow will necessitate the selection of strut configuration such that the pitch [P] (one complete rotation of the strut around its axis per unit length, regardless of its radial cross-section configuration), as shown in FIGS. 1A and 1B, is adjusted depending on the selected width-to-height ratio of the streamlined strut profile. Likewise, in embodiments where the cross-section of the strut is streamlined, the radius (r) may be changed to maintain the same pressure distribution across the strut in the flow direction regardless of the lumen diameter in which the stent of the invention is implanted.

The three-dimensional assembly of the cross-section depends in one embodiment upon the macro-configuration of the struts, e.g., whether the stent is a coil, lattice, expanded overlapping rings, etc. For each however, the elements of strut cross-sectional streamlining described above will be optimized taking into account the final deployment of the struts in relation to the flow direction. This is accomplished by design optimization to determine the full 3-dimensional configuration of the stent based upon the constraints of cross-sectional strut design described herein.

In a second embodiment, discussions herein referring to the surface contour or cross-sectional shape of stent 1A and 1B refer not to the surface contour or cross-sectional shape of the individual struts of the stent but rather to the overall cross-sectional shape of the stent, namely the entire coil element or lattice pattern or other configuration collectively. In this embodiment, each strut of the stent contributes to the overall shape of the stent cross-sectional shape.

The flow field upstream and downstream of the nonstreamlined strut cross-sections is governed in certain embodiments, by recirculating flow. Such flow structure is the characteristics observed in another embodiment, in atherosusceptible regions of the arterial tree. In one embodiment, regions in the arterial tree where the flow has to turn sharply promote fluid flow separation away from the wall resulting in the development of vortices, secondary motions, and flow reversal. This phenomenon occurs in one embodiment, in regions like the carotid sinus where rapid expansion of the arterial geometry promotes flow separation. In this region, the flow cannot laminarly follow the vessel geometry resulting in a regional separation of flow with the development of secondary motions, such as helical motions accompanied by flow reversal. This specific region correlates with intimal thickening and the presence of plaque. In another embodiment, the carotid flow-divider, an area where the flow is predominantly unidirectional and attached to the wall, is relatively spared of intimal thickening. Other regions where the flow separates due to the arterial geometry are in one embodiment, the inner wall of the aortic arch, which exhibits high endothelial expression of ICAM-1 and VCAM-1 and atherosusceptible and procoagulant phenotypes, or in another embodiment the proximal renal ostium, which exhibits greater propensity towards the development of lesions as opposed to the distal side of the renal ostium where flow separation is unlikely to occur. In one embodiment, after stent implantation, the newly formed wall composed of the blood vessel and struts creates a boundary with a sudden change in direction when transitioning from the top to the side surface of the strut where the blood flow can separate when trying to change directions rapidly. In one embodiment, using the stents described herein flow reversal or separation are minimized to the point where no secondary motions occur.

As blood cells travel tangentially to the strut surface they are exposed in one embodiment to large shearing forces (FIG. 15). In the case of platelets, high shear forces result in activation and release of thromboxane A2 (TXA2) in one embodiment, or adenosine diphosphate (ADP) in another embodiment, or both, two potent mediators of platelet aggregation. The numerical simulations of rectangular stent struts provided in the examples show shear rate values above 3000 s⁻¹. Platelet activation occurs in certain embodiments, at shear rate levels as low as 2200 s⁻¹. In another embodiment, erythrocytes release about 2% of their ADP at shear rate values of 5680 s⁻¹, resulting in sufficient amounts of ADP to induce platelet aggregation. In one embodiment, ADP induces shape change in platelets, and promotes platelet aggregation and surface expression of fibrin receptors. Activated platelets or erythrocytes exposed to high shear forces while being convected along the strut surface have the potential to enter the recirculation zone. The recirculation zone is likely populated by lower amounts of PGI₂ and NO, potent inhibitors of platelet aggregation. Under normal conditions the interaction between PGI₂ and TXA₂ represents a balanced system that controls platelet function by inhibiting platelet aggregation in the absence of local injury. However, current commercial (nonstreamlined) stent struts can establish flow conditions that lead to an unbalanced state favoring platelet aggregation and thrombogenesis. If thrombogenesis occurs, shed procoagulant microparticles can become entrapped in the recirculation zone further accelerating the thrombus growth rate. FIGS. 11a and 11b summarize the hemostatic balance in arteries and outline the predicted procoagulant consequences of stent-related flow separation.

The ideal surface to inhibit thrombogenesis consists in one embodiment, of an intact endothelium in an atheroprotective flow environment, such as those provided by the streamlined stents described herein. Endothelial cells normally express an anticoagulant phenotype. When a stent is implanted, there is a high probability of partial endothelial denudation that tips the balance towards a procoagulant surface environment. A high flow (high shear) rate environment provides in another embodiment, a superior condition for endothelialization when compared to a low flow (low shear) rate environment. In another embodiment, the strut leading and trailing edge angles influence endothelialization rates; wherein smaller slopes being more favorable for endothelialization. Depending on the geometric characteristics of the stent strut cross-section, the local flow environment promotes in one embodiment, or retard, or inhibit endothelialization in other embodiments. A nonstreamlined strut cross-section will promote flow separation and promote development of recirculation zones yielding low shear rates (FIG. 11b). In contrast, FIGS. 11c and 11d illustrate how the streamlined strut geometry provided herein will minimize or avoid the generation of a low flow velocity environment distal and proximal to the strut and will promote faster endothelialization of the strut surface and the neighboring vessel wall.

Shear stress levels over the surface of a nonstreamlined or thick strut can reach very high levels that can also be detrimental to endothelialization. In one embodiment, the yield stress corresponding to endothelial denudation is about 38 Pa, which is about 13 times typical coronary arterial values, but plausible in stenotic regions or over the top surface of stent struts that are exposed to much higher blood flow velocities than those present in the near-wall region (FIG. 15). In contrast, the streamlined struts provided herein avoid the development of low shear stress sites in the near vicinity as well as local high shear stress peaks that can inhibit endothelialization.

The numerical simulation results provided herein are supported by clinical results from the ISAR-STEREO Trial. In the ISAR-STEREO Trial, patients were randomly implanted with two types of bare metal stents with similar architectures, material properties, and strut widths (100 μm), but with different strut thicknesses (50 μm versus 140 μm). The angiographic restenosis rates decreased by 42% in the group of patients that received the 50 μm versus the thicker (140 μm) strut group. Given that the only variable that was changed in the clinical trial was the strut thickness, it can be concluded that the stent strut geometry affects the restenosis rate, which is a marker of clinical success. As strut thickness increases for a nonstreamlined geometry the flow disturbances increase nonlinearly, generating flow conditions that have been linked to an atherosusceptible flow environment in which there are low levels of NO and PGI2, molecules linked to inhibition of smooth muscle cell (SMC) proliferation and migration [in addition to their anti-coagulant properties. SMCs and their secreted extracellular matrix are the predominant elements of neointimal hyperplasia, which is principally responsible for vessel restenosis after stent implantation. As the thickness of a nonstreamlined stent strut decreases, the tissue area that will experience flow recirculation will decrease resulting in a lower probability of restenosis, a scenario consistent with the results of the ISAR-STEREO Trial. In one embodiment, increasing the flow rate (shear stress) regresses restenosis.

In one embodiment, when the hemodynamics of the local environment is taken into account in combination with aerodynamic theory, the streamlined stent cross-section provided herein is incorporated into a stent. A streamlined design minimize in one embodiment, or eliminate the flow recirculation zone in another embodiment, establishing an atheroprotective and anticoagulant flow environment conducive to endothelialization of the strut surface and adjacent vessel wall, optimal conditions for clinical success (FIG. 11). A large decrease in flow separation results from a modest degree of streamlining. Thus changes in the geometry of relatively thick struts lead in one embodiment to improved hemodynamics, an attractive compensation as the material strength limits of strut thinness are reached. In the case of BMS, streamlining disclosed herein improves in another embodiment the hemodynamics during the critical period of several weeks before the stent is overgrown by neointima. In the case of DES, the struts remain on the artery surface and protrude into the flow for long periods due to their antiproliferative therapeutic properties that prevent neointimal overgrowth. A nonstreamlined strut protruding into the flow field promotes the creation of recirculation zones, which are nidi for thrombogenesis and possibly high shear stress peaks over the surface that can activate platelets. The streamlined DES provided herein, is less likely to create the conditions necessary for the development of recirculation zones and high shear stress peaks over the surface, even at higher strut thicknesses than a thinner nonstreamlined strut, resulting in faster healing of the vessel and less probability of stent thrombosis.

Implantation of the stent described herein requires no special equipment or procedures beyond those already well known in the art for implantation of stents within coronary or peripheral arteries.

The following examples are presented in order to more fully illustrate the preferred embodiments of the invention. They should in no way be construed, however, as limiting the broad scope of the invention.

### EXAMPLES

### Materials and Methods

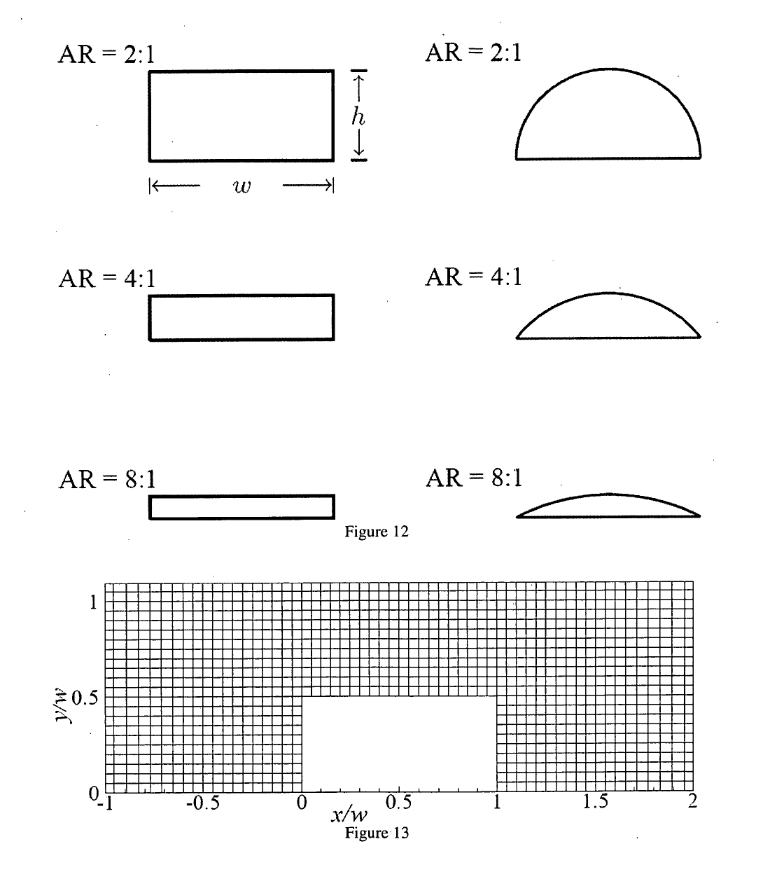

A set of numerical simulations was conducted to elucidate the role of stent strut geometries and their effects on the local hemodynamic conditions in a generic section of a coronary artery (FIG. 1). Rather than investigating the flow field about specific commercial stent strut geometries, which are predominantly rectangular and nonstreamlined (Table 1), we investigated representative geometries of commercial stents along with aerodynamically inspired designs. Six different stent strut geometries were studied and the surrounding flow fields analyzed in order to establish a relationship between the strut geometry and resulting flow characteristics (FIG. 12). The continuity and momentum equations were solved using the Fluent Computational Fluid Dynamics (CFD) software (Ansys Inc., Lebanon, N.H., USA). Pressure fields, shear stress and shear rate distributions are presented for each case studied.

TABLE 1
Several commercial stents and their basic geometric characteristics.

| | | | Strut | Drug |
|---|---|---|---|---|
| | | Approximate | Thickness | Coating |
| Stent | Company | Geometry | (μm) | (μm) |
| Cypher | Cordis (J&J) | Trapezoid | 140 | 12.6 |
| Taxus | Boston | Trapezoid | 132 | 16 |
| Express | Scientific | | | |
| Endeavor | Medtronic | Circular | 91 | 5.3 |
| Xience V | Abbott | Rectangular | 81 | 7.6 |
| Taxus | Boston | Rectangular | 97 | 15 |
| Liberté | Scientific | | | |

### Fluid Domain and Conditions.

The geometrical model used for the simulations consists of a 19.2 mm long, L, and 3 mm in diameter, D, straight rigid tube (FIG. 1). The effects of elasticity in the vessel are small so the assumption of rigid tube flow is reasonable. A series of six independent rings represents the architecture of the simplified stent. The number of rings coincides with commercial stents used for shorter lesions, but higher numbers of rings are present in stents used to treat longer lesions. The flow is assumed to be axisymmetric to minimize the computational cost; therefore the results are characteristic for any streamwise plane. This case represents a relatively straight region of a vessel away from bifurcations or branches where secondary motions can be present.

The cross-sectional geometry of the struts consists of rectangles and circular arcs with varying aspect ratios, AR, of width to height, w:h, from 2:1, 4:1, and 8:1 (FIG. 12). The width, w was kept constant at 200 ìm for all cases while h was decreased from 100 ìm to 50 ìm to 25 ìm for the 2:1, 4:1, and 8:1 aspect ratio cases, respectively. The interstrut spacing was set to 10w, which is in the interstrut distance range for typical commercial stents. Also, the first and last strut were located more than 1D streamwise into the flow field to ensure that any numerical error perturbation present at the inlet or outlet did not affect the local flow field.

The inlet boundary condition consisted of a parabolic velocity profile with a mean velocity Ū, equal to 0.3812 m/s, which corresponds to the peak diastolic coronary blood flow velocity. The parabolic velocity profile is described by the following equation;

$$u ( r ) = 2 \overline{U} [ 1 - {( \frac{2 r}{D} )}^{2} ] \qquad ( 1 )$$

Where Ū is the mean velocity and r is the radial spatial coordinate. The outlet boundary condition was defined by a constant pressure condition. The no-slip condition was applied to all solid surfaces and a symmetry condition was applied to the centerline of the vessel. The dynamic viscosity, μ, and density, ρ, of the blood used for the numerical simulations were 0.00304 kg/m·s and 1060 kg/m3, respectively. Coronary arteries are characterized by high blood flow rates and medium-size lumen diameters yielding relatively high shear stresses that inhibit the aggregation of blood components, which is a common phenomenon at lower shear rates. Also, larger blood cells tend to populate the inner core of the flow due to the Magnus effect resulting in a plasma-rich near-wall region. These characteristics of coronary arterial flow makes modeling blood as a Newtonian fluid an adequate approximation, since non-Newtonian effects are observed predominantly in much smaller vessels than the coronary arteries where cell-cell interactions are not negligible and the length scale of the cells is of the order of the vessel diameter. The assumption of Newtonian fluid can affect the computed dimensions of recirculation zones, which are populated by slower moving cells. The flow conditions for these simulations are limited to a single time point within the unsteady cardiac cycle, which coincides with the maximum flow rate during diastole and yields a Reynolds number based upon the vessel diameter, Re_{D}=ρUD/μ, of approximately 400, which is the peak Reynolds number for the cardiac cycle. Due to the unsteady nature of cardiac blood flow, the simulations provide quantitative results for the maximum flow rate instance and a qualitative representation of the rest of the cycle.

### Governing Equations

The governing continuity and momentum equations for a steady, Newtonian, incompressible, viscous, axisymmetric flow in cylindrical coordinates are given by Eqs. (2), (3), and (4),

$$\begin{matrix}\frac{1}{r} \frac{\partial ( rv )}{\partial r} + \frac{\partial u}{\partial x} = 0 , & ( 2 ) \\ ρ ( v \frac{\partial v}{\partial r} + u \frac{\partial v}{\partial x} ) = - \frac{\partial p}{\partial r} + μ [ \frac{1}{r} \frac{\partial }{\partial r} ( r \frac{\partial v}{\partial r} ) + \frac{{\partial }^{2} v}{\partial {x}^{2}} - \frac{v}{{r}^{2}} ] , & ( 3 ) \\ ρ ( v \frac{\partial u}{\partial r} + u \frac{\partial u}{\partial x} ) = - \frac{\partial p}{\partial x} + μ [ \frac{1}{r} \frac{\partial }{\partial r} ( r \frac{\partial u}{\partial r} ) + \frac{{\partial }^{2} u}{\partial {x}^{2}} ] . & ( 4 )\end{matrix}$$

where u and v are the axial and radial velocity components, respectively. The independent variables x and r are the streamwise and radial spatial coordinates, respectively. The pressure is denoted by p. The wall shear stress is defined by the following,

$$\begin{matrix}{τ}_{w} = μ ( \frac{\partial v}{\partial r} + \frac{\partial u}{\partial x} ) \\ = μ \dot{γ} ,\end{matrix} \qquad ( 5 )$$

where \dot{γ}• is the shear rate.

### Comutational Fluid Dynamics

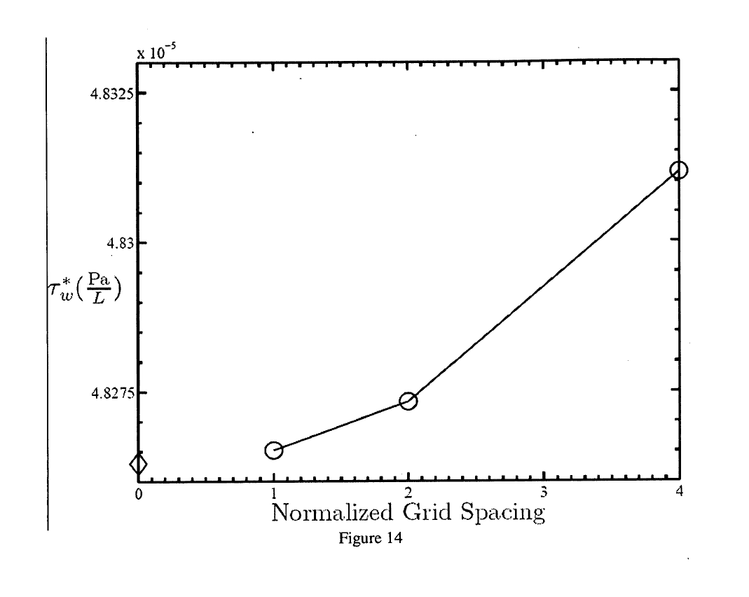

The governing equations were solved for each flow field using second-order finite difference solvers. A mesh convergence study was conducted by increasing the number of nodes approximately by a factor of 2 in each dimension. The approximate total number of nodes for the three different meshes was 81 000, 323 000, and 1 320 000. The mesh was composed of quadrilateral elements for the rectangular strut flow field, and quadrilateral and triangular elements for the circular arc struts. The total number of nodes surrounding the struts increased by approximately 3.88 and 3.99 times from mesh 1 to mesh 2 and from mesh 2 to mesh 3, respectively. The meshes were generated manually with a higher concentration of nodes near the wall and stent struts to resolve the larger near-wall gradients (FIG. 13). FIG. 14 shows a typical grid convergence plot for wall shear stress per unit length (τ*_{w}) variation over a strut as a function of grid spacing. Each iteration was run until the solution converged. The convergence criterion was reached when the residuals of the independent variables had decreased by at least 14 orders of magnitude. Furthermore, the set of iterations showed that the converged solutions were grid independent. Refining the grid spacing further would only decrease the error by less than 3% as shown by the theoretical τ*_{w} value calculated using Richardson extrapolation.

### Example 1

### Blood Flow across the Struts is Different between Streamlined and Nonstreamlined Stents

The fluid flow domain shown in FIG. 1 was studied using CFD to better understand the effects of six streamlined and nonstreamlined stent strut geometries with varying aspect ratios, AR, in a blood vessel (FIG. 12). The coordinates on the plots shown in this section have been modified for ease of presentation without any modifications to the data. Axes corresponding to distance have been nondimensionalized by w and the location of the leading edge of the third strut has been redefined as x/w=0. In this study there is no focus on the strut-to-strut flow field variations, but on a representative case. Qualitatively the flow field about a strut is similar to that of its neighboring struts, but quantitative differences can be observed since the effects due to the presence of neighboring struts compounds as the flow travels downstream in the blood vessel. The effect is greater for thicker struts.

### Example 2

### Pressure Field

FIGS. 4 and 5 shows the nondimensionalized pressure field in the vicinity of the struts with streamlines in the foreground. The pressure was nondimensionalized by dividing the static pressure by the dynamic pressure,

$${p}^{*} = \frac{p}{\frac{1}{2} ρ {\overline{U}}^{2}} .$$

A higher pressure region is present for each case on the upstream side of the strut. The pressure gradient weakens as the height, h, decreases. The flow fields along the top surfaces of the struts experience a pressure decrease as x/w increases, but upstream of the struts the pressure increases as it approaches x/w=0. The upstream influence of the strut increases as the height of the strut increases. Since the flow studied is laminar and steady, the superimposed streamlines in FIGS. 4 and 5 correspond to the path a fluid element traveled in space. A significant recirculation region, as denoted by the streamlines, is present both upstream and downstream of the 2:1 rectangular geometry (FIG. 4a). Similar results are observed on the upstream and downstream side of the 4:1 and 8:1 rectangular struts (FIGS. 4b and 4c). For the circular arc stent strut geometries, flow separation is only observed in the vicinity of the 2:1 aspect ratio (FIG. 5a). The 4:1 and 8:1 aspect ratio circular arc struts do not demonstrate any flow separation for the flow conditions studied.

### Example 3

### Separation Zone Cross-Sectional Area

Table 2 shows the upstream and downstream separation areas normalized by the separation area of the rectangular 8:1 aspect ratio strut. The upstream separation zones corresponding to the rectangular 4:1 and 2:1 cases, increased 3.8 and 8.4 times, respectively, when compared to that of the 8:1 aspect ratio strut. Correspondingly, the up stream separation zone for the 2:1 circular arc increased 20%. The downstream separation area increased nonlinearly from 5.7 to 42.2 times for the 4:1 and 2:1 rectangular struts, respectively. The downstream separation zone for the 2:1 circular arc increased about 14.4 times with respect to the downstream separation zone of the rectangular 8:1 aspect ratio strut, which is a significantly larger increase than the increase observed for the upstream side, but significantly lower than that observed for the 2:1 rectangular strut. The upstream separation zone for the rectangular 4:1 case is larger than that for the 2:1 circular strut, but the opposite is true for the downstream side. Although sharing a similar aspect ratio, the upstream and downstream separation areas of the 2:1 circular arc are reduced by 98% and 66%, respectively when compared with the 2:1 rectangular stent strut.

TABLE 2
Separation zones upstream and downstream
of nonstreamlined strut geometries.

| Geometry | Upstream Area | Downstream Area |
|---|---|---|
| Rectangular 2:1 | 8.4 | 42.4 |
| Rectangular 4:1 | 3.8 | 5.7 |
| Rectangular 8:1 | 1.0 | 1.0 |
| Circular Arc 2:1 | 1.2 | 14.4 |

The areas are normalized by the separation area corresponding to upstream and downstream separation areas of the 8:1 Rectangular case

### Example 4

### Separation Distance

Table 3 shows the separation distance corresponding to the axial length of the separation zone. The separation distance increased as h increased. The upstream separation distances are 0.223w, 0.145w, and 0.074w for the 2:1, 4:1, and 8:1 rectangular stent strut geometries, respectively. The 2:1 rectangular stent strut exhibits two distinct separation zones, with separation lengths of 0.002w and 0.845w for the smaller recirculation zone closest to the strut/vessel corner and a larger one that extents further downstream and surrounds the smaller counter-rotating vortex, respectively. The downstream separation length for the 4:1 and 8:1 rectangular struts are 0.257w and 0.094w, respectively. The downstream separation distance for the 2:1 AR is decreased by approximately 44% when the geometry is simply changed from a rectangular to a circular arc cross-section while keeping the same aspect ratio.

TABLE 3
Separation length upstream and downstream
of nonstreamlined strut geometries.

| Geometry | Upstream Distance | Downstream Distance |
|---|---|---|
| Rectangular 2:1 | 0.223 | 0.002 |
| | | 0.845 |
| Rectangular 4:1 | 0.145 | 0.257 |
| Rectangular 8:1 | 0.074 | 0.094 |
| Circular Arc 2:1 | 0.08 | 0.469 |

The distance is normalized by the strut width, w.

### Example 5

### Wall Shear Stress and Shear Rate

Wall shear stress, τ_{w}, and shear rate, \dot{γ}, distributions over the rectangular struts and in the near vicinity are shown in FIG. 15a as a function of x/w. Due to the proportional relationship between wall shear stress and shear rate, τ_{ω}=μ\dot{γ}, the following discussion although focused on the wall shear stress distributions is applicable to the shear rate distributions. As x/w→0⁻ and x/w→1⁺, τ_{w}→0 for all rectangular cases in FIG. 15a. The effective region over which τ_{w}≈0 decreases as h decreases and is always confined immediately upstream or downstream of the strut. The effects are more noticeable in the downstream side. FIG. 15b shows the wall shear stress distribution over and in the near vicinity of the circular arc strut geometries. The wall shear stress levels for the 2:1 circular arc follow the trends observed for the rectangular designs; low shear stress levels dominate the vicinity of the struts, but without distinct peaks in the shear stress distribution (FIG. 15). The regions of low shear stress coincide with the separation zones that in the case of the 4:1 and 8:1 circular arc designs are negligible. The shear stress distribution for the rectangular designs has two peaks at the upstream and downstream corners where the flow velocity increases significantly over a small distance. Removing the abrupt change in geometry encountered in a rectangular strut and replacing it with gradual geometric changes, such as those observed in the circular arcs, inhibits the development of regions of concentrated high shear stress. The wall shear stress values for the upstream peaks corresponding to the rectangular stent geometries increased 54% and 101% when h was doubled and quadrupled from 25 μm to 50 μm and 25 μm to 100 μm, respectively (Table 4). The increase in τ_{w} for the downstream peaks was less with a 27% and 32% increase when AR was varied from 8:1 to 4:1 and 8:1 to 2:1, respectively. Due to the eventual favorable pressure gradient over the forward face of the circular arc struts, the flow accelerated and resulted in a gradual increase of the shear stress values. The maximum shear stress over the circular arc struts increased by 53% and 158% when AR was varied from 8:1 to 4:1 and 8:1 to 2:1, respectively. The maximum shear stress values corresponding to the circular arcs were approximately 50% lower than the corresponding rectangular geometries (FIG. 15).

TABLE 4
Shear stress and shear rates for streamlined
and nonstreamlined stent strut designs

| | τ_{w} (Pa) | τ_{w} (Pa) | \grave{γ} (s⁻¹) | \acute{γ} (s⁻¹) |
|---|---|---|---|---|
| | Upstream | Downstream | Upstream | Downstream |
| Geometry | Peak | Peak | Peak | Peak |
| Rectangular 2:1 | 32.6 | 13.7 | 10723.7 | 4506.6 |
| Rectangular 4:1 | 16.2 | 10.4 | 5328.9 | 3421.1 |
| Rectangular 8:1 | 10.5 | 8.2 | 3453.9 | 2697.4 |

| Geometry | τ_{w} (Pa)-Maximum. | \acute{γ} (s⁻¹)-Maximum |
|---|---|---|
| Circular Arc 2:1 | 14.6 | 4802.6 |
| Circular Arc 4:1 | 8.7 | 2861.8 |
| Circular Arc 8:1 | 5.7 | 1871.7 |

### Example 6

### Streamlined Geometries

For the Reynolds number and flow conditions studied a streamlined geometry was defined as one that inhibits flow separation due to gradual changes in the slope over the surface. While substantial reduction of flow separation is accomplished with a 2:1 circular arc geometry, the flow about the circular arc struts of AR values 4:1 and 8:1 does not separate and exhibits gradual variations in the shear rate and shear stress distributions (FIG. 4a-4c). The 4:1 and 8:1 circular arc stent strut geometries meet the streamlined body definition (FIG. 5a-5c).

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A stent providing attached or minimally separated blood flow therethrough, said stent comprising one of more struts each having a leading section and a trailing section in the direction of blood flow through said stent, said strut comprising:
a cross-sectional geometry longitudinally disposed thereon for blood flow thereover, comprising a leading region and a trailing region with a continuous surface contour with varying slope throughout,
wherein the leading subsection introduces a favorable pressure gradient over the leading section of the strut and affects a directional change in blood flow, the trailing section affects a directional change in blood flow, and a midsection disposed therebetween, thereby providing attached or minimally separated blood flow over each said stent strut and throughout the length of the stent.

2. The stent of claim 1 wherein said strut has a length defined as the distance from said leading edge to said trailing edge and a height defined as the distance between an upper and a lower surface thereof.

3. The stent of claim 2 wherein the ratio of said width to said height is greater than 2:1

4. The stent of claim 2 wherein the ratio of said width to said height is between about 2:1 and about 8:1.

5. The stent in claim 2, wherein the strut is extended to double peak height symmetric to a line extended from the leading edge to the trailing edge.

5. The stent of claim 1 wherein said strut has an outer surface whose contour in the longitudinal direction substantially corresponds to the radius of curvature of a lumen within which said stent is implanted.

6. The stent of claim 1 wherein linker struts extend upstream and downstream within said stents to connect elements of a lattice stent design, said linker strut having an outer surface whose contour in the transverse direction substantially corresponds to the radius of curvature of a lumen within which said stent is implanted.

7. A method of ensuring flow over a stent that is substantially free of disturbed flow, comprising:
implanting in a predetermined location in a coronary or peripheral artery a stent providing attached or minimally separated blood flow therethrough, said stent comprising one of more struts each having a leading section and a trailing section in the direction of blood flow through said stent, said strut comprising:
a cross-sectional geometry longitudinally disposed thereon for blood flow thereover, comprising a leading section and a trailing section with a continuous surface contour with varying slope throughout,
wherein the leading subsection introduces a favorable pressure gradient over the leading section of the strut affecting a directional change in blood flow without resulting in blood flow separation, the trailing section affects a directional change in blood flow without inducing blood flow separation, and a midsection disposed therebetween, thereby providing attached or minimally separated blood flow over each said stent strut and throughout the length of the stent.

8. The method of claim 6, whereby said strut has a length defined as the distance from said leading edge to said trailing edge and a height defined as the distance between an upper and a lower surface thereof.

9. The method of claim 8 wherein the ratio of said width to said height is greater than about 2:1

10. The method of claim 8 wherein the ratio of said width to said height is from 2:1 to about 8:1 or greater.

11. A drug eluting system for ensuring an attached flow over a drug-eluting stent that is substantially free of disturbed flow, comprising
the drug-eluting stent of claim 1; and
means for implanting the stent.

12. A bare metal system for ensuring an attached flow over a bare metal stent that is substantially free of disturbed flow, comprising
the bare metal stent of claim 1; and
means for implanting the stent.

13. A degradable system for ensuring an attached flow over a degradable stent that is substantially free of disturbed flow, comprising the degradable stent of claim 1; and means for implanting the stent.
